(19) United States
(12) Patent Application Publication
(10) Pub. No.: US 2003/0053966 A1
(43) Pub. Date: Mar. 20, 2003

(54) Insoluble organic UV screening agents of aryl vinylene ketone type, and cosmetic use thereof

(76) Inventor: Herve Richard, Villepinte (FR)

Correspondence Address: NIXON & VANDERHYE P.C., 8th Floor, 1100 North Glebe Rd., Arlington, VA, 22201-4714, US

(21) Appl. No.: 10/191,111

(22) Filed: Jul. 10, 2002

(30) Foreign Application Priority Data
Jul. 18, 2001 (FR) 01 09634

(51) Int. Cl. A61K 7/42

(52) U.S. Cl. 424/59

(57) ABSTRACT

The invention relates to insoluble organic compounds containing one or more aryl vinylene ketone groups, for screening out UV radiation, in the form of fine particles with an average size of between 10 nm and 5 μm, and also to their use for protecting the skin, the hair or materials that are sensitive to UV radiation. The said compounds may be obtained by a process of grinding coarse particles in the presence of one or more suitable surfactants.

The present invention relates to insoluble organic compounds of aryl vinylene ketone type for screening out UV radiation, to cosmetic compositions containing them and to their use as agents for screening out UV radiation for protecting materials that are sensitive to UV radiation, the skin or the hair or for controlling the colour of the skin.

It is known that light radiation with wavelengths of between 280 nm and 400 nm permit tanning of the human epidermis and that light rays with wavelengths between 280 and 320 nm, known as UV-B rays, cause skin burns and erythema which can harm the development of a natural tan. For these reasons, and also for aesthetic reasons, there is a constant demand for means for controlling this natural tanning in order thus to control the colour of the skin; this UV-B radiation should thus be screened out.

It is also known that UV-A rays, with wavelengths between 320 and 400 nm, which cause browning of the skin, are liable to induce adverse changes therein, in particular in the case of sensitive skin or skin which is continually exposed to solar radiation. UV-A rays in particular cause a loss of elasticity of the skin and the appearance of wrinkles, leading to premature ageing of the skin. They promote triggering of the erythemal reaction or amplify this reaction in certain individuals and may even be the cause of phototoxic or photoallergic reactions. Thus, for aesthetic and cosmetic reasons such as the conservation of the skin's natural elasticity, for example, an increasingly large number of people wish to control the effect of UV-A rays on their skin. It is thus desirable also to screen out UV-A radiation.

A large number of compounds have already been proposed as sunscreens, essentially in the form of soluble organic screening agents or insoluble mineral compounds. These screening agents must be able to absorb or block out the harmful rays of the sun while at the same time remaining harmless to the user.

In this regard, and in order to limit any possible risks of skin allergy associated with the use of soluble organic screening agents, an increasingly large number of insoluble mineral compounds such as zinc oxide or titanium oxide have been used for some time. However, these mineral compounds may have the drawback of being sensitive to solar radiation. Moreover, for equivalent amounts, these mineral compounds are less effective in UV protection than organic screening agents.

A certain number of organic compounds containing an aryl vinylene ketone group have already been proposed as sunscreens. Examples that may be mentioned include the benzylidene camphors described in patent FR 2 528 420, the benzylidene-nor-camphors described in patent EP 0 693 471, the benzylidene ketotricyclodecanes described in patent EP 0 694 521, the benzylidene cineoles described in the 16th IFSCC Congress of New York (1990), the benzylidene quinuclidinones described in patents EP 0 576 974 and EP 0 714 880, the styryl ketones described in patent JP 04 134 042, the para-xylidene ketones described in patent JP 04 134 041, the alpha-benzylidene ketones described in patent JP 04 134 043, the alpha-acetyl cinnamates described in patent JP 11 080 091, the benzylidene cycloalkanones described in patent FR 2 395 023, the benzylidene hydantoins described in patent JP 01 158 090, the benzylidene furanones described in patents EP 0 390 683 and FR 2 638 354, the chalcones described in patents FR 2 555 167 and FR 2 608 150, and the alpha-pyrones described in patent JP 04 290 882.

However, none of these documents mentions the use of these screening agents in the form of insoluble particles of very small size, i.e. of the order of one micron or less than one micron in size.

Documents WO 95/22959, WO 97/03643, WO 99/66896 and WO 00/78277 also disclose organic UV screening agents or mixtures thereof that are insoluble or difficult to dissolve, which may comprise, inter alia, derivatives of the type such as oxalanilide, triazine, triazole, vinyl amide, cinnamamide, benzimidazole-sulphonic, cinnamic acid, diphenylacrylate, camphor, etc., micronized in the form of particles with an average size of between 0.01 and 2 μm. Some of these screening agents, in particular the benzimidazole-sulphonic derivatives, show variations in solubility at high temperature which lead to a substantial recrystallization in the support. Such a phenomenon may be reflected by a loss of efficacy of the screening agents and a deterioration of the cosmetic properties of the compositions containing them.

After considerable research conducted in the field of photoprotection, the Applicant has discovered novel insoluble organic compounds containing one or more aryl vinylene ketone groups that are capable of absorbing both in the UV-A range and in the UV-B range, which are in the form of fine particles with an average size of between 10 nm and 5 μm, for effectively protecting the skin, the lips or the hair and also other photosensitive materials against the unfavourable effects of UV radiation.

For the purposes in which the present invention is used, the term “aryl vinylene ketone” encompasses all aryl vinylene derivatives containing a carbonyl alpha to the double bond and not containing an amide group.

For the purposes of the present invention, the expression “insoluble compound” means a compound with a solubility in water of less than 0.1% by weight and a solubility of less than 1% by weight in most organic solvents, for instance liquid paraffin, fatty alkyl benzoates and fatty acid triglycerides, for example Miglyol® 812 sold by the company Dynamit Nobel. This solubility, defined at 70° C. as the amount of product in solution in the solvent at equilibrium with an excess of solid in suspension, may readily be evaluated in the laboratory.

These insoluble organic compounds make it possible to overcome the drawbacks of the soluble or insoluble organic screening agents and the insoluble mineral screening agents of the prior art. These materials are sunscreens that are more effective than the mineral screening agents of the prior art, since they combine two screening mechanisms, namely the absorption of UV radiation by the organic chromophores and the scattering-reflection of light by the surfaces of the particles. They are thus entirely suitable for the preparation of cosmetic compositions intended for protecting the skin and the hair against solar radiation. Furthermore, besides their screening and scattering properties, the insoluble organic compounds according to the invention have good chemical and photochemical stability.

One subject of the present invention is, consequently, insoluble organic compounds for screening out UV radiation, of the aryl vinylene ketone type corresponding to one of the formulae (I) and (II) below, and which are in the form of particles with an average size of between 10 nm and 5 μm.

A subject of the invention is also cosmetic compositions containing at least one insoluble organic compound of formula (I) or (II), in the form of particles with an average size of between 10 nm and 5 μm.

A subject of the invention is also the use of these insoluble compounds as agents for screening out UV-A and UV-B radiation, for example for protecting materials that are sensitive to UV radiation, in particular to solar radiation, for controlling the colour of the skin or for preparing medicinal products for preventing the harmful effects of UV-A and UV-B radiation.

The insoluble organic compounds of the aryl vinylene ketone type for screening out UV radiation, with an average size of between 10 nm and 5 μm, are chosen from those corresponding to one of the formulae (I) and (II) below:

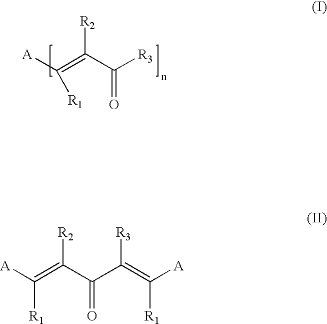

in which:

n=1 or 2,

A, in formula (I) when n=1 or in formula (II), is an aryl radical chosen from formulae (a) to (d) below, or in formula (I) when n=2, is a radical chosen from formulae (e) to (h) below:

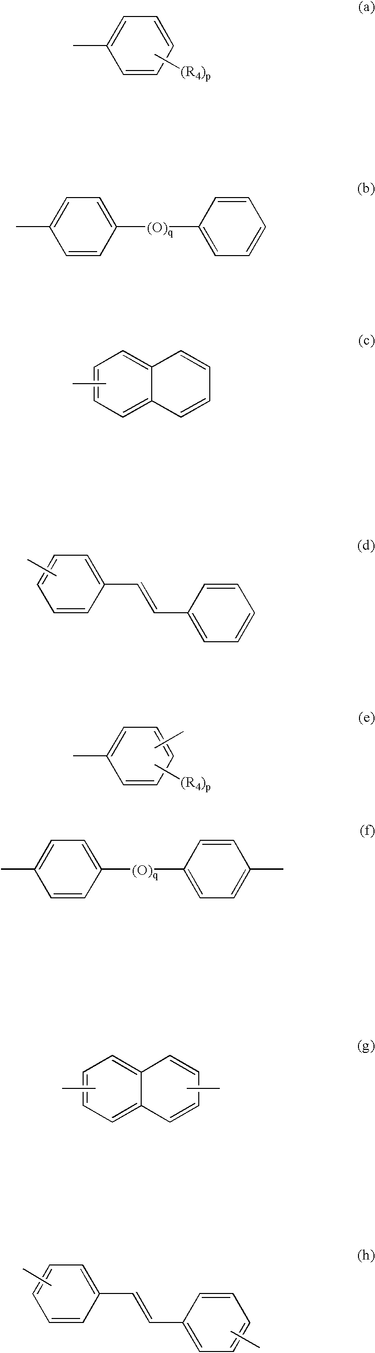

in which:

each of the symbols R₄ independently represents an OH group, a halogen atom, a linear or branched C₁₋₆ alkyl group optionally containing a silicon atom, a linear or branched C₁₋₆ alkoxy group optionally containing a silicon atom, a linear or branched C₁₋₅ alkoxycarbonyl group, or a linear or branched C₁₋₆ alkylsulphonamide group optionally containing a silicon atom or an amino acid function,

p represents an integer between 0 and 4 inclusive,

q represents 0 or 1,

R₁ represents hydrogen or an OH group,

R₂ represents hydrogen, a linear or branched C₁₋₆

alkyl group optionally containing a silicon atom,

a cyano group, a C₁₋₆ alkylsulphonyl group or a phenylsulphonyl group,

R₃ represents a linear or branched C₁₋₆ alkyl group optionally containing a silicon atom or a phenyl group that can form a bicycle and optionally substituted with one or two radicals R₄,

or R₂ and R₃ together form a monocyclic, bicyclic or tricyclic C₂₋₁₀ hydrocarbon residue optionally interrupted with one or more nitrogen, sulphur and oxygen atoms and possibly containing another carbonyl, and optionally substituted with a linear or branched C₁-C₈ alkylsulphonamide group, and optionally containing a silicon atom or an amino acid function; on condition that, when n=1, R₂ and R₃ do not form a camphor nucleus.

As examples of insoluble compounds of formula (I) in which n=1, for screening out UV radiation, with an average particle size of between 10 nm and 5 μm, mention may be made of the following families:

Styryl ketone (Kao JP 04 134 042) such as 1-(3,4-dimethoxyphenyl)-4,4-dimethylpent-1-en-3-one:

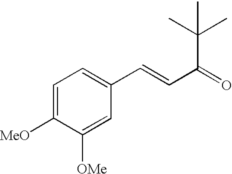

Benzylidene cineole (E. Mariani et al, 16th IFSCC Congress, New York (1990)) such as 1,3,3-trimethyl-5-(4-methoxybenzylidene)-2-oxabicyclo[2.2.2]octan-6-one:

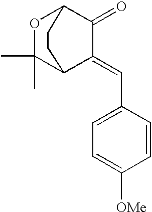

Benzylidene chromanone (Kao JP 04 134 043) such as 3-(4-methoxybenzylidene)-2,3,4a,8a-tetra-hydrochromen-4-one:

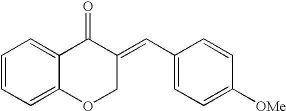

Benzylidene thiochromanone (Kao JP 04 134 043) such as 3-(4-methoxybenzylidene)-2,3,4a,8a-tetrahydrochromene-4-thione:

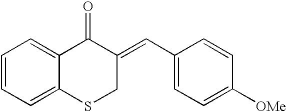

Benzylidene quinuclidinone (Merck EP 0 576 974) such as 4-methoxybenzylidene-1-azabicyclo-[2.2.2]octan-3-one:

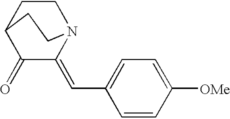

Benzylidene cycloalkanone (Henkel FR 2 395 023) such as 2-(4-methoxybenzylidene) cyclopentanone and 2-(4-methoxybenzylidene)cyclohexanone:

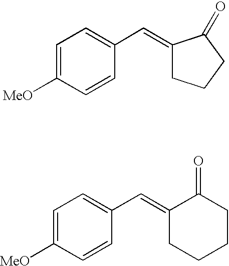

Benzylidene hydantoin (Ajinomoto JP 01 158 090) such as 5-(3,4-dimethoxybenzylidene)imidazolidine-2,4-dione:

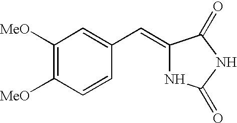

Benzylidene indanone (Kao JP 04 134 043) such as 2-(4-methoxybenzylidene)indan-1-one:

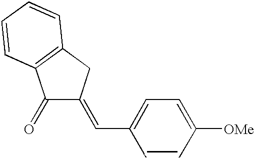

Benzylidene tetralone (Kao JP 04 134 043) such as 2-(4-methoxybenzylidene)-3,4-dihydro-2H-naphthalen-1-one:

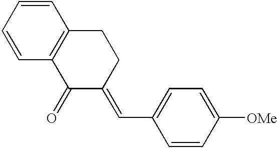

Benzylidene furanone (L'Oréal EP 0 390 683) such as 4-(4-methoxybenzylidene)-2,2,5,5-tetramethyldihydrofuran-3-one:

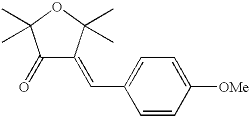

Benzylidene benzofuranone (Kao JP 04 134 041) such as 2-benzylidenebenzofuran-3-one:

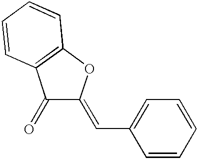

Benzylidene indanedione such as 2-(3,5-di-tertbutyl-4-hydroxybenzylidene)indan-1,3-dione:

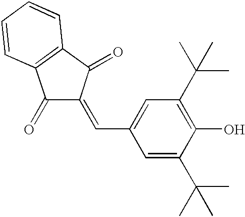

Benzylidene benzothiofuranone (Kao JP 04 134 043) such as 2-benzylidenebenzo[b]thiophen-3-one:

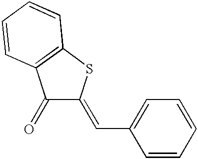

Benzylidene barbituric such as 5-(4-methoxybenzylidene)-1,3-dimethylpyrimidine-2,4,6-trione:

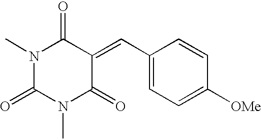

Benzylidene pyrazolone such as 4-(4-methoxybenzylidene)-5-methyl-2-phenyl-2,4-dihydropyrazol-3-one:

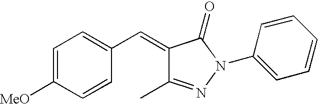

Benzylidene imidazolone such as 5-(4-methoxybenzylidene)-2-phenyl-3,5-dihydroimidazol-4-one:

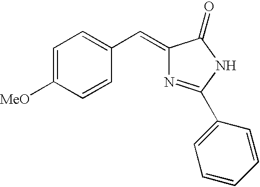

Chalcone, such as 1-(2-hydroxy-4-methoxyphenyl)-3-phenylpropenone:

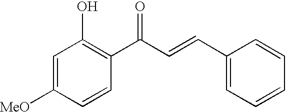

Benzylidene one (screening tautomeric form of dibenzoylmethanes; L'Oréal FR 2 506 156) such as 3-hydroxy-1-(2-hydroxy-4-methoxyphenyl)-3-phenylpropenone:

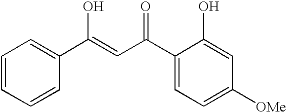

As examples of insoluble compounds of formula (I) in which n=2, for screening out UV radiation, with an average particle size of between 10 nm and 5 μm, mention may be made of the following families:

Phenylenebis(methylidene-nor-camphor) (Merck EP 0 693 471) such as 1,4-phenylene-bis{3-methylidenebicyclo[2.2.1]heptan-2-one}:

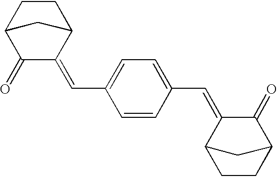

Phenylenebis(methylidenecamphor) (L'Oréal FR 2 528 420) such as 1,4-phenylenebis{3-methylidene-1,7,7-trimethylbicyclo[2.2.1]-heptan-2-one}:

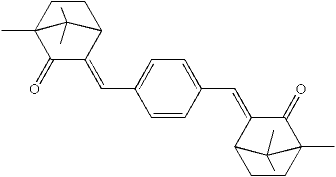

or 1,3-phenylenebis{3-methylidene-1,7,7-trimethylbicyclo[2.2.1]heptan-2-one}:

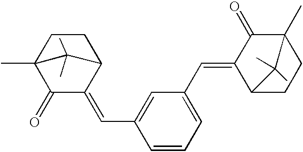

Phenylenebis(methylidenecamphorsulphonamide) (L'Oréal FR 2 529 887) such as 1,4-phenylenebis-(ethyl or 2-ethylhexyl 3,3′-methylidenecamphor-10,10′-sulphonamide):

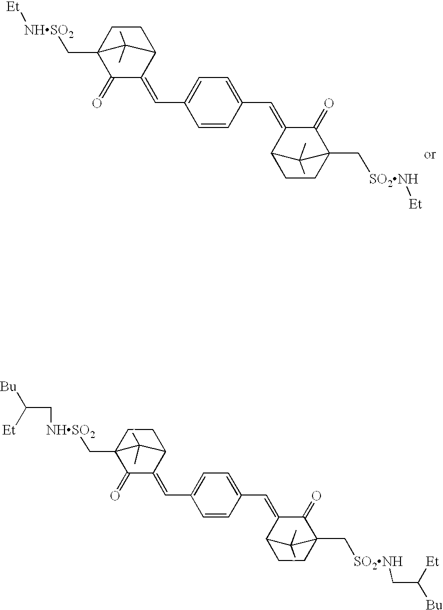

Phenylenebis(methylidene cineole) (E. Mariani et al., 16^{th} IFSCC Congress, New York (1990)) such as 1,4-phenylenebis{5-methylidene-3,3-dimethyl-2-oxabicyclo[2.2.2]octan-6-one}:

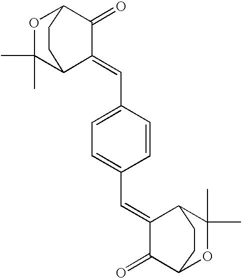

Phenylenebis(methylidene ketotricyclodecane) (Merck EP 0 694 521) such as 1,4-phenylenebis-(octahydro-4,7-methano-6-inden-5-one):

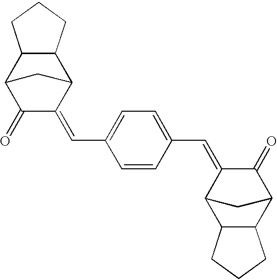

Phenylenebis (alkylene ketone) (Kao JP 04 134 041) such as 1,4-phenylenebis(4,4-dimethylpent-1-en-3-one):

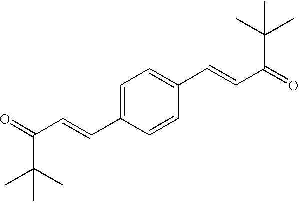

Phenylenebis(methylidene furanone) (L'Oréal FR 2 638 354) such as 1,4-phenylene-bis(4-methylidene-2,2,5,5-tetramethyldihydrofuran-3-one):

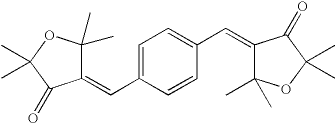

Phenylenebis(methylidene quinuclidinone) (Merck EP 0 714 880) such as 1,4-phenylenebis{2-methylidene-1-azabicyclo[2.2.2]octan-3-one}:

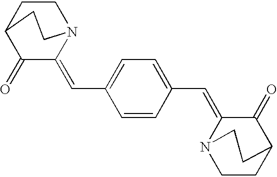

As compounds of formula (II), mention may be made of the following families:

bis(benzylidene)cycloalkanone such as 2,5-dibenzylidenecyclopentanone:

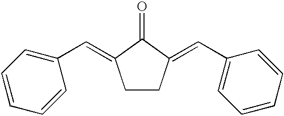

gamma-pyrone (Kao JP 04 290 882) such as 2,6-bis(3,4-dimethoxyphenyl)pyran-4-one:

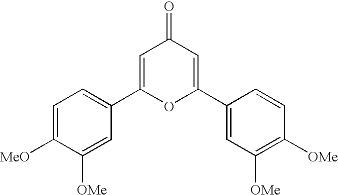

Among these insoluble organic compounds for screening out UV radiation, the ones that are most particularly preferred are the compounds of formula (I) in which n=2.

As mentioned above, the average size of the insoluble particles of the organic compounds described above is between 10 nm and 5 μm.

The particles preferably have an average size of between 10 nm and 2 μm and more preferably between 20 nm and 2 μm.

In general, the average size of the particles will correspond to the number-average diameter of the distribution.

The mean particle size may be determined by any standard method such as optical methods (quasi-elastic scattering or laser scattering), centrifugation methods or methods of microscopic visualization and image analysis.

The insoluble organic screening agents according to the invention may be provided in the form of particles having the desired average size by any suitable means, such as dry grinding or grinding in a solvent medium, screening, atomization, micronization, spraying or any chemical process such as precipitation by emulsion or dilution.

The insoluble organic screening agents according to the invention may be obtained in particular by a process of grinding coarse particles in the presence of one or more suitable surfactants for improving the dispersion of the particles thus obtained in cosmetic formulations.

One example of a process for reducing the particle size of insoluble organic screening agents is described in documents WO 95/22959 and WO 97/03643. The grinding machine used according to these documents may be a jet mill, a ball mill, a vibration mill or a hammer mill and preferably a mill with a high stirring speed or an impact mill and more particularly a rotary ball mill, a vibrating mill, a tube mill or a rod mill.

The UV-screening insoluble organic compounds may be used in cosmetic compositions, in particular compositions for protecting the human epidermis or the hair, antisun compositions or makeup products.

These cosmetic compositions generally contain from 0.1% to 15% by weight and preferably from 0.2% to 10% by weight, relative to the total weight of the cosmetic composition, of insoluble organic compounds for screening out UV radiation.

The cosmetic compositions, and in particular antisun compositions, of the present invention may also contain one or more additional organic sunscreens that are active in the UV-A and/or UV-B range, other than the insoluble screening agents described above.

These sunscreens may be chosen especially from cinnamic derivatives, dibenzoylmethane derivatives; salicylic derivatives; benzylidenecamphor derivatives; triazine derivatives such as those described in patents or patent applications U.S. Pat. No. 4,367,390, EP 0 863 145, EP 0 517 104, EP 0 570 838, EP 0 796 851, EP 0 775 698, EP 0 878 469, EP 0 933 376, EP 0 507 691, EP 0 507 692, EP 0 790 243, EP 0 944 624 and U.S. Pat. No. 4,724,137; benzophenone derivatives; β,β′-diphenylacrylate derivatives; phenylbenzimidazole derivatives; anthranilic derivatives; imidazoline derivatives; methylene-bis(hydroxyphenylbenzotriazole) derivatives such as those described in patents or patent applications U.S. Pat. No. 5,237,071, U.S. Pat. No. 5,166,355, GB 2 303 549, DE 19726184 and EP 0 893 119; p-aminobenzoic acid derivatives; screening hydrocarbon polymers and screening silicones such as those described especially in patent application WO 93/04665; dimers derived from α-alkylstyrene as described in patent application DE 198 55 649; 4,4-diarylbutadienes as described in patent applications EP 0 967 200 and DE 197 55 649; and also mixtures of these screening agents.

Examples of such additional UV-A active and/or UV-B active sunscreens that may be mentioned include the following compounds, denoted by their INCI name, and also mixtures thereof:

Para-Aminobenzoic Acid Derivatives:

PABA,

Ethyl PABA,

Ethyl dihydroxypropyl PABA,

Ethylhexyl dimethyl PABA sold in particular under the name “Escalol 507” by ISP,

Glyceryl PABA,

PEG-25 PABA sold under the name “Uvinul P25” by BASF,

Salicylic Derivatives:

Homosalate sold under the name “Eusolex HMS” by Rona/EM Industries,

Ethylhexyl salicylate sold under the name “Neo Heliopan OS” by Haarmann and Reimer,

Dipropylene glycol salicylate sold under the name “Dipsal” by Scher,

TEA salicylate sold under the name “Neo Heliopan TS” by Haarmann and Reimer,

Dibenzoylmethane Derivatives:

Butyl methoxydibenzoylmethane sold in particular under the trade name “Parsol 1789” by Hoffmann LaRoche,

Isopropyldibenzoylmethane,

Cinnamic Derivatives:

Ethylhexyl methoxycinnamate sold in particular under the name “Parsol MCX” by Hoffmann LaRoche,

Isopropyl methoxycinnamate,

Isoamyl methoxy cinnamate sold under the trade name “Neo Heliopan E 1000” by Haarmann and Reimer,

Cinoxate,

DEA methoxycinnamate,

Diisopropyl methylcinnamate,

Glyceryl ethylhexanoate dimethoxycinnamate

β,β′-diphenylacrylate Derivatives:

Octocrylene sold in particular under the trade name “Uvinul-539” by BASF

Benzophenone Derivatives:

Benzophenone-1 sold under the trade name “Uvinul 400” by BASF,

Benzophenone-2 sold under the trade name “Uvinul D50” by BASF,

Benzophenone-3 or Oxybenzone, sold under the trade name “Uvinul M-40” by BASF,

Benzophenone-4 sold under the trade name “Uvinul MS-40” by BASF,

Benzophenone-5,

Benzophenone-6 sold under the trade name “Helisorb 11” by Norquay,

Benzophenone-8 sold under the trade name “Spectra-Sorb UV-24” by American Cyanamid,

Benzophenone-9 sold under the trade name “Uvinul DS-49” by BASF,

Benzophenone-12

Benzylidenecamphor Derivatives:

Benzylidenecamphorsulphonic acid manufactured under the name “Mexoryl SL” by Chimex,

Camphor benzalkonium methosulphate manufactured under the name “Mexoryl SO” by Chimex,

Terephthalylidenedicamphorsulphonic acid manufactured under the name “Mexoryl SX” by Chimex,

Polyacrylamidomethylbenzylidenecamphor manufactured under the name “Mexoryl SW” by Chimex,

Phenylbenzimidazole Derivatives:

Phenylbenzimidazolesulphonic acid sold in particular under the trade name “Eusolex 232” by Merck,

Benzimidazilate sold under the trade name “Neo Heliopan AP” by Haarmann and Reimer,

Triazine Derivatives:

Anisotriazine sold under the trade name “Tinosorb S” by Ciba-Geigy,

Ethylhexyltriazone sold in particular under the trade name “Uvinul T150” by BASF,

Diethylhexylbutamidotriazone sold under the trade name “Uvasorb HEB” by Sigma 3V,

2,4,6-Tris(diisobutyl 4′-aminobenzalmalonate) s-triazine

Phenylbenzotriazole Derivatives:

Drometrizole trisiloxane sold under the name “Silatrizole” by Rhodia Chimie,

Methylenebis(benzotriazolyl)tetramethylbutylphenol sold in solid form under the trade name MIXXIM BB/100 by Fairmount Chemical, or in micronized form as an aqueous dispersion under the trade name Tinosorb M by Ciba Specialty Chemicals

Anthranilic Derivatives:

Menthyl anthranilate sold under the trade name “Neo Heliopan MA” by Haarmann and Reimer,

Imidazoline Derivatives:

Ethylhexyldimethoxybenzylidenedioxoimidazoline propionate,

Benzalmalonate Derivatives:

Polyorganosiloxane containing benzalmalonate functions, sold under the trade name “Parsol SLX” by Hoffmann LaRoche.

The organic UV screening agents that are more particularly preferred are chosen from the following compounds:

Ethylhexyl salicylate,

Butyl methoxydibenzoylmethane,

Ethylhexyl methoxycinnamate,

Octocrylene,

Phenylbenzimidazolesulphonic acid,

Terephthalylidenedicamphorsulphonic acid,

Benzophenone-3,

Benzophenone-4,

Benzophenone-5,

Benzimidazilate,

Anisotriazine,

Ethylhexyltriazone,

Diethylhexylbutamidotriazone,

2,4,5-tris(diisobutyl 4′aminobenzalmalonate) 5-triazine,

Methylenebis(benzotriazolyl)tetramethylbutylphenol,

Drometrizole trisiloxane, and mixtures thereof.

The cosmetic compositions according to the invention may also contain agents for artificially tanning and/or browning the skin (self-tanning agents) such as dihydroxyacetone (DHA).

The cosmetic compositions according to the invention may also contain one or more mineral pigments and in particular metal oxide nanopigments, that may be coated or uncoated, such as, for example, nanopigments of titanium oxide in amorphous or crystalline (rutile and/or anatase) form, of iron oxide, of zinc oxide, of zirconium oxide or of cerium oxide. These nanopigments have a mean particle size of between 5 nm and 100 nm and preferably between 10 nm and 50 nm, and are all known UV-photoprotective agents.

These nanopigments may be coated with known coating agents such as, for example, alumina and/or aluminium stearate.

Such coated or uncoated nanopigments are described, for example, in patent applications EP-A-0 518 772 and EP-A-0 518 773.

The cosmetic compositions may also contain adjuvants of common formulation such as fatty substances, physiologically acceptable organic solvents, silicones, surfactants, anionic, cationic, nonionic or amphoteric polymers or mixtures thereof, thickeners, antioxidants, opacifiers, stabilizers, antifoams, fragrances, preserving agents, fillers, sequestering agents, propellants, pH modifiers and colorants, and mixtures thereof.

They may also contain one or more cosmetic active principles chosen, for example, from softeners, hydroxy acids, vitamins, moisturizers, emollients, free-radical scavengers, substance P antagonists, anti-inflammatories and mixtures of these compounds.

The fatty substances may consist of an oil or a wax or mixtures thereof, and they also comprise fatty acids, fatty alcohols and fatty acid esters. The oils may be chosen from animal, plant, mineral and synthetic oils and especially from liquid petroleum jelly, liquid paraffin, silicone oils, that may be volatile or non-volatile, isoparaffins, polyolefins, fluoro oils and perfluoro oils. Similarly, the waxes may be chosen from animal, fossil, plant, mineral and synthetic waxes that are known per se.

Among the organic solvents that may be mentioned are lower alcohols and polyols such as ethanol, isopropanol, propylene glycol, glycerol and sorbitol.

The thickeners may be chosen especially from guar gums and celluloses, that may be modified or unmodified, such as hydroxypropyl guar gum, methylhydroxyethylcellulose, hydroxypropylmethylcellulose or hydroxyethylcellulose.

Needless to say, the person skilled in the art will take care to select this or these optional additional compound(s) and/or the amounts thereof such that the advantageous properties intrinsic to the invention, and in particular the insolubility of the agents for screening out UV radiation, are not adversely affected by the envisaged addition(s).

The compositions of the invention may be prepared according to techniques that are well known to those skilled in the art, in particular those intended for preparing emulsions of oil-in-water or water-in-oil type.

This composition may be in particular in the form of a simple or complex emulsion (O/W, W/O, O/W/O or W/O/W emulsion) such as a cream, a milk, a gel or a cream-gel, a powder or a solid tube, and may optionally be packaged as an aerosol and be in the form of a mousse or a spray.

When it is an emulsion, the aqueous phase of this emulsion may comprise a nonionic vesicular dispersion prepared according to known processes (Bangham, Standish and Watkins, J. Mol. Biol. 13, 238 (1965), FR 2 315 991 and FR 2 416 008).

The cosmetic composition of the invention may be used as a composition for protecting the human epidermis or the hair against ultraviolet rays, as an antisun composition or as a makeup product.

When the cosmetic composition according to the invention is used for protecting the human epidermis against UV rays, or as an antisun composition, it may be in the form of a suspension or a dispersion in solvents or fatty substances, in the form of a nonionic vesicular dispersion or in the form of an emulsion, preferably of oil-in-water type, such as a cream or a milk, or in the form of an ointment, a gel, a cream-gel, a solid tube, a stick, a powder, an aerosol mousse or a spray.

When the cosmetic composition according to the invention is used for protecting the hair against UV rays, it may be in the form of a shampoo, a lotion, a gel, an emulsion or a nonionic vesicular dispersion and may constitute, for example, a rinse-out composition, to be applied before or after shampooing, before or after dyeing or bleaching, before, during or after permanent-waving or straightening the hair, a styling or treating lotion or gel, a blow-drying or hairsetting lotion or gel, or a composition for permanent-waving, straightening, dyeing or bleaching the hair.

When the composition is used as a makeup product for the eyelashes, the eyebrows or the skin, such as an epidermal treatment cream, a foundation, a tube of lipstick, an eyeshadow, a face powder, a mascara or an eyeliner, it may be in solid or pasty, anhydrous or aqueous form, for instance oil-in-water or water-in-oil emulsions, nonionic vesicular dispersions or suspensions.

As a guide, for the antisun formulations in accordance with the invention, which contain a support of oil-in-water emulsion type, the aqueous phase generally represents from 50% to 95% by weight and preferably from 70% to 90% by weight, relative to the total formulation, the oily phase represents from 5% to 50% by weight and preferably from 10% to 30% by weight, relative to the total formulation, and the (co)emulsifier(s) represent(s) from 0.5% to 20% by weight and preferably from 2% to 10% by weight, relative to the total formulation.

The insoluble screening agents of the present invention may also be used for protecting materials, for instance organic or mineral glasses or plastics, that are sensitive to UV radiation, in particular to solar radiation. The particles are then applied onto or incorporated into the material to be protected in an effective amount.

A subject of the invention is also a non-therapeutic process for protecting the skin and/or the hair against ultraviolet radiation, in particular solar radiation, which consists in applying to the skin or the hair an effective amount of the cosmetic composition defined above, or of a compound of formula (I) or (II) as defined above.

Finally, a subject of the invention is a non-therapeutic process for controlling the variation in the colour of the skin caused by UV radiation, which consists in applying to the skin an effective amount of the cosmetic composition defined above, or of a compound of formula (I) or (II) as defined above.

The examples that follow illustrate the invention.

### EXAMPLES OF COMPOUNDS

### Example 1

Synthesis of 1,4-phenylenebis(ethyl 3,3′-methylidenecamphor-10,10′-sulphonamide)

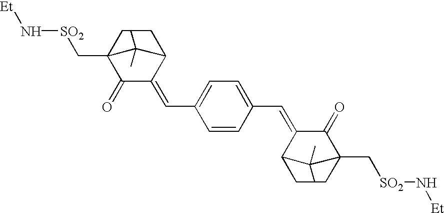

The disodium salt of 1,4-phenylenebis(3,3′-methylidenecamphor-10,10′-sulphonic acid) (12.1 g, 0.02 mol) is suspended in 50 ml of dry DMF. The temperature rises to 32° C. Ethylamine (4.5 g, 0.1 mol) is then added, followed by triethylamine (4.9 g, 0.044 mol). The mixture is stirred for 1 hour. The cooled reaction mixture is poured into 100 ml of water. The solid obtained is filtered off, washed with water and then with an acetone/water mixture to give the 1,4-phenylenebis(ethyl 3,3′-methylidenecamphor-10,10′-sulphonamide) (5 g, yield: 80%) in the form of a white powder (m.p. 165° C.):

UV (solution in DMSO/CH₂Cl₂): λₘₐₓ=342 nm, εₘₐₓ=40350, E1%=654

Elemental analysis for C₃₂H₄₄N₂O₆S₂ Calculated: C62.31 H7.19 N 4.54 O15.56 S10.40 Found: C62.78 H7.14 N4.21 O16.12 S10.62

### Example 2

Synthesis of 1,4-phenylenebis(2-ethylhexyl 3,3′-methylidenecamphor-10,10′-sulphonamide)

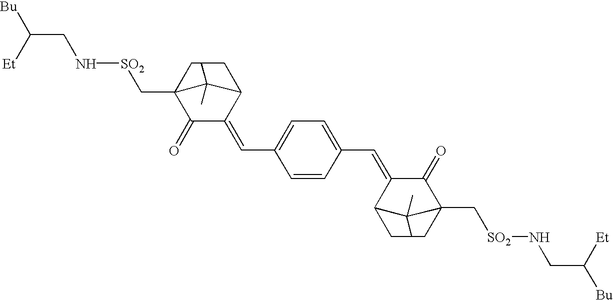

The disodium salt of 1,4-phenylenebis(3,3′-methylidenecamphor-10,10′-sulphonic acid) (12.1 g, 0.02 mol) is suspended in 50 ml of dry DMF. The temperature rises to 32° C. 2-Ethylhexylamine (5.6 g, 0.044 mol) is then added, followed by triethylamine (4.9 g, 0.044 mol). The mixture is stirred for 1 hour. The resulting mixture is extracted with dichloromethane and the organic phase is washed with water and dried, and the solvent is evaporated off. The very thick oil obtained is crystallized from ethanol to give the 1,4-phenylenebis(2-ethylhexyl 3,3′-methylidenecamphor-10,10′-sulphonamide) (30 g, yield: 64%) in the form of a white powder (m.p. 165° C.):

UV (solution in CH₂Cl₂): λₘₐₓ=342 nm, εₘₐₓ=41 000, E1%=522

Elemental analysis for C₄₄H₆₈N₂O₆S₂ Calculated: C67.35 H8.67 N3.51 O12.25 S8.16 Found: C67.40 H8.55 N3.21 O12.67 S7.70

### Example 3

Synthesis of 1,3-phenylenebis(3-methylidenecamphor)

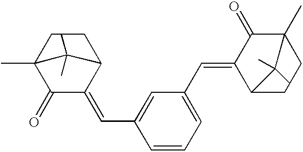

Camphor (33.5 g, 0.22 mol) and potassium tert-butoxide powder (24.7 g, 0.22 mol) are placed in 250 ml of dry toluene in a round-bottomed flask equipped with Dean Stark apparatus. The mixture is brought to reflux and left at this temperature for 1 hour. The resulting mixture is cooled to about 40° C. and isophthalaldehyde (13.4 g, 0.1 mol) dissolved in 200 ml of dry toluene is then added dropwise over 1 hour. The mixture is maintained for 3 hours 30 minutes at reflux with removal of the water formed. The mixture is cooled and poured into ice containing 25 ml of concentrated hydrochloric acid. The organic phase is separated out, washed with water, dried over sodium sulphate and filtered, and the solvent is evaporated off. The mass obtained is purified on a column of silica (eluent: CH₂Cl₂) and then recrystallized from cyclohexane to give the 1,3-phenylenebis(3-methylidenecamphor) (10.4 g, yield 25%) in the form of a white powder (m.p. 152° C.).

UV (solution in CHCl₃): λₘₐₓ=295 nm, εₘₐₓ=41 500, E1%=1 040

Elemental analysis for C₂₈H₃₄O₂ Calculated: C83.54 H8.51 O7.95 Found: C83.48 H8.50 O8.25

### Example 4

Synthesis of 1,4-phenylenebis(methylidene ketotricyclodecane)

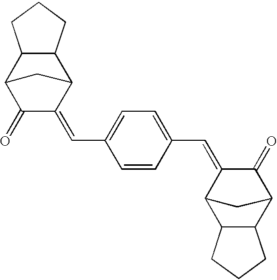

8-Oxotricyclo[5.2.1.0^{2,6}]decane (22 g, 0.15 mol) and 200 ml of toluene are introduced into a round-bottomed flask equipped with a condenser, a dropping funnel, a thermometer and a nitrogen inlet, and the mixture is brought to reflux. Sodium methoxide (9.72 g, 0.18 mol) is added. The mixture is stirred for 20 minutes. para-Terephthalaldehyde (8.04 g, 0.06 mol) as a solution in a mixture of 50 ml of toluene and 10 ml of methanol is then added dropwise over 2 hours. The mixture is refluxed for 3 hours. The resulting mixture is cooled and poured into water. The organic phase is dried and concentrated. The crude product obtained is recrystallized from 500 ml of a ⅔ dichloromethane/isopropyl ether mixture to give the 1,4-phenylenebis(methylidene ketotricyclodecane) (4.2 g, yield 81%) in the form of a white powder. (m.p.=235-236° C.).

UV (solution in CHCl₃): λₘₐₓ=340 nm, εₘₐₓ=43 100, E1%=1 080.

Elemental analysis for C₂₈H₃₀N₂O₂ Calculated: C84.38 H7.59 O8.03 Found: C84.19 H7.68 O8.14.

### Example 5

Synthesis of 1,4-phenylenebis(3-methylidenecamphor)

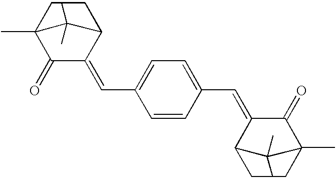

Camphor (66.98 g, 0.44 mol) and sodium methoxide powder (23.76 g, 0.44 mol) are placed in 100 ml of dry toluene in a fully equipped round-bottomed flask. The mixture is brought to reflux and maintained at this temperature for 1 hour. Terephthalaldehyde (26.8 g, 0.2 mol) dissolved in a hot mixture of 100 ml of dry toluene, 10 ml of pyridine and 16 ml of methanol is then added dropwise over 2 hours. The methanol is distilled off and the temperature is maintained at 110° C. for 8 hours. The reaction mixture is poured into ice and extracted with diethyl ether. The solid is recrystallized from diethyl ether to give, after drying, the 1,4-phenylenebis(3-methylidenecamphor) (23 g, 13% yield) in the form of a white powder (m.p. 234° C.).

UV (solution in CHCl₃): λₘₐₓ=337 nm, εₘₐₓ=38 720, E1%=960.

Elemental analysis for C₂₈H₃₄O₂ Calculated: C83.54 H8.51 O7.95 Found: C83.54 H8.56 O7.91

Preparation of an Aqueous Dispersion of the Screening Agent:

A 50% dispersion in water of the insoluble screening agent in the presence of 1% of alkyl polyglucoside (APG) sold by the company Henkel under the brand name Plantaren is prepared.

Preparation of the Predispersion:

Small fractions of powder are introduced into the prehomogenized water+APG mixture in a 50 ml beaker.

The mixture is agitated by ultrasound for 45 minutes to wet the powder thoroughly.

Grinding:

Equipment used: Recht MM200 ball mill sold by the company Recht, 25 ml bowl, 2 steel balls 2 cm in diameter.

The predispersion obtained above is introduced into the bowl of the mill containing the balls. Grinding is performed for 1 hour at a stirring frequency of 15.

Characterization:

The dispersion obtained using the compound prepared according to Example 5 of the invention is characterized using a Mastersizer 2000 granulometer sold by the company Malvern.

The number-average diameter distribution is: d=0.77 μm for the product ground for one hour in the equipment used.

### Example 6

Synthesis of (2,3,5,6-tetramethyl)-1,4-phenylenebis-(3-methylidenecamphor)

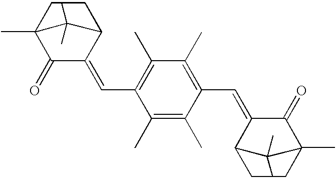

Camphor (40 g, 0.264 mol), sodium hydroxide pellets (9.6 g, 0.24 mol) and 2,3,5,6-tetramethyl terephthalaldehyde (22.82 g, 0.12 mol) are placed in 200 ml of dry toluene in a fully equipped round-bottomed flask. The mixture is brought to reflux and left at this temperature for 96 hours. The reaction mixture is poured into ice and extracted with chloroform. The organic phase is washed with water and dried over sodium sulphate. After filtration, drying and cutting through silica (eluent: toluene), the (2,3,5,6-tetramethyl)-1,4-phenylenebis(3-methylidenecamphor) (14.3 g, 26% yield) is obtained in the form of a white powder.

UV (solution in CH₂Cl₃): λₘₐₓ=286 nm, εₘₐₓ=12 200, E1%=270.

Elemental analysis for C₃₂H₄₂O₂ Calculated: C83.79 H9.23 O7.05 Found: C83.59 H9.24 O6.98

### Example 7

Synthesis of 1,4-phenylenebis(2-methylidene quinuclidinone)

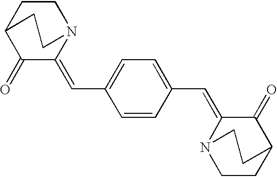

Quinuclidinone hydrochloride (32 g, 0.2 mol) is introduced into 100 ml of water in a fully equipped round-bottomed flask. A solution of sodium hydroxide (64 g in 200 ml of water) is added thereto. The mixture is brought to 80° C. under nitrogen and terephthalaldehyde (12.06 g, 0.09 mol) dissolved in 150 ml of hot ethanol is added over 30 minutes. A yellow precipitate appears. The mixture is refluxed for 4 hours. The resulting mixture is cooled and filtered, and the solid is washed with water. After drying under vacuum, the 1,4-phenylenebis(2-methylidene quinuclidinone) (21.5 g, 69% yield) is obtained in the form of a pale yellow powder (m.p.>270° C.).

UV (solution in DMSO): λₘₐₓ=346 nm, εₘₐₓ=29 600, E1%=850 λₘₐₓ=364 nm, εₘₐₓ=25 000, E1%=717.

Elemental analysis for C₂₀H₁₂N₂O₂ Calculated: C75.83 H6.94 N8.04 O9.18 Found: C75.45 H7.08 N8.00 O9.37.

### Example 8

Synthesis of 2,5-bis[4-(3-trimethylsilanylpropyloxy)-benzylidene]cyclopentanone

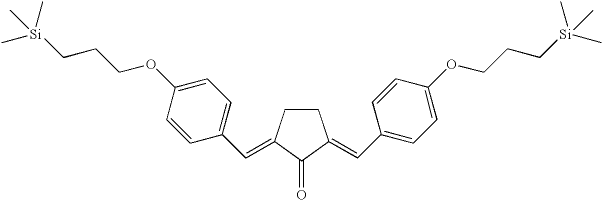

a) First Step: Preparation of 4-(3-trimethylsilanylpropyloxy)benzaldehyde:

3-Chloropropyltrimethylsilane (33.14 g, 0.22 mol) is added dropwise over 10 minutes to a mixture of 4-hydroxybenzaldehyde (24.4 g, 0.2 mol) and potassium carbonate (30.4 g, 0.22 mol) in 150 ml of dry DMF brought to 120° C. under nitrogen. The mixture is stirred for 2 hours 30 minutes at 120-130° C. The reaction mixture is cooled and poured into ice-water. The aqueous phase is extracted 3 times with dichloromethane. The organic phases are dried over sodium sulphate and concentrated under vacuum. After distillation under vacuum (0.2 mmHg), 40.5 g (yield: 86%) of 4-(3-trimethylsilanylpropyloxy)benzaldehyde are obtained in the form of a colourless oil distilling at 110-114° C., and used without further purification in the following step.

b) Second Step: Preparation of the Derivative of Example 8:

4.2 g of sodium hydroxide (0.105 mol) dissolved in 22 ml of water are added dropwise over 15 minutes at 35° C. with stirring to a mixture of the above product (26.2 g, 0.105 mol) and cyclopentanone (4.2 g, 0.05 mol) in 200 ml of methanol, under nitrogen. The mixture is refluxed for 6 hours. The resulting mixture is cooled and the yellow precipitate obtained is filtered off and washed thoroughly with water. After drying, 25 g (yield: 96%) of the derivative of Example 8 are obtained in the form of a yellow solid.

Melting point: 205-207° C.

UV (solution in CHCl₃): λₘₐₓ=391 nm, εₘₐₓ=49 100, E1%=940.

Elemental analysis for C₃₁H₄₄O₃Si₂ Calculated: C71.49 H8.51 Si10.78 Found: C71.15 H8.78 Si10.47.

### Example of a Cosmetic Composition Oil-in-Water Cream

| | | |
|---|---|---|
| 80/20 mixture of cetylstearyl alcohol and | 7 | g |
| of oxyethylenated (33 EO) cetylstearyl | | |
| alcohol (Sinnowa ® AO - Henkel) | | |
| Mixture of glyceryl monostearate and | 2 | g |
| distearate (Cerasynt ® SD-V ISP) | | |
| Cetyl alcohol | 1.5 | g |
| Polydimethylsiloxane (Dow Corning 200 | 1.5 | g |
| Fluid ® - Dow Corning) | | |
| C12/C15 Alkyl benzoates (Witconol TN - | 15 | g |
| Witco) | | |
| Ground 1,4-phenylenebis(3-methylidene- | 10 | g |
| camphor) as an aqueous 50% dispersion, | | |
| according to Example 5 | | |
| Glycerol | 20 | g |
| Preserving agents | qs | |
| Demineralized water | qs 100 | g |

The fatty phase and the aqueous phase are heated separately to 70° C. and the oily phase is then introduced into the aqueous phase with stirring. Stirring is continued for about 15 minutes and the composition is allowed to cool with gentle stirring until it has returned to room temperature.

The efficacy of this composition is assessed in vitro using an SPF 290 spectroradiometer sold by the company Optometrics and according to the method of Diffey and Robson (B. L. Diffey et al., J. Soc. Cosmet. Chem., 40, 127-133 (1989)) on a quartz plate coated with a Transpore® tape. The sun protection factor (SPF) is equal to 3.0±0.3.

1. Insoluble organic compound of aryl vinylene ketone type for screening out UV radiation, which is in the form of particles with an average size of between 10 nm and 5 μm, and corresponding to one of the formulae (I) and (II) below: 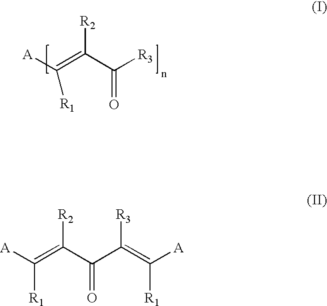
in which:
n=1 or 2,
A, in formula (I) when n=1 or in formula (II), is an aryl radical chosen from formulae (a) to (d) below, or in formula (I) when n=2, is a radical chosen from formulae (e) to (h) below: 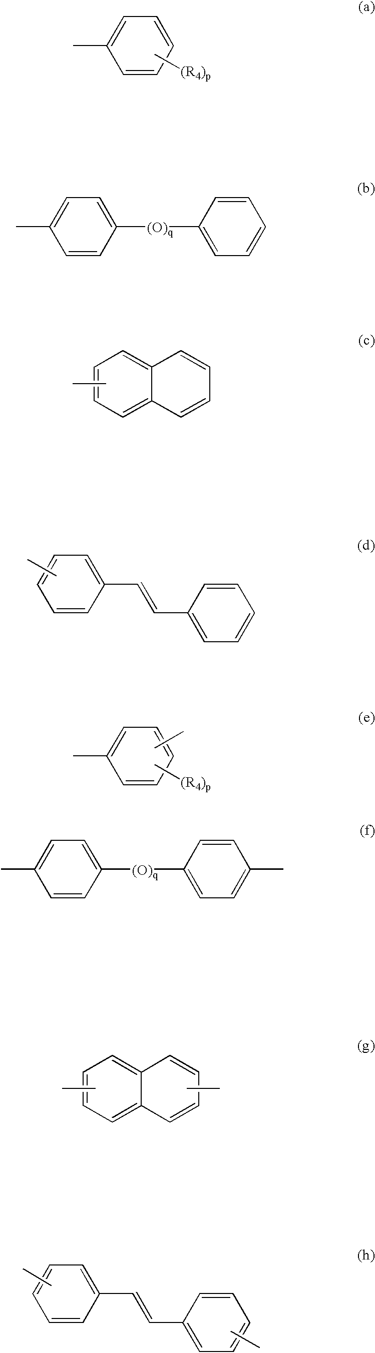
in which:
each of the symbols R₄ independently represents an OH group, a halogen atom, a linear or branched C₁₋₆ alkyl group optionally containing a silicon atom, a linear or branched C₁₋₆ alkoxy group optionally containing a silicon atom, a linear or branched C₁₋₅ alkoxycarbonyl group, or a linear or branched C₁₋₆ alkylsulphonamide group optionally containing a silicon atom or an amino acid function,
p represents an integer between 0 and 4 inclusive,
q represents 0 or 1,
R₁ represents hydrogen or an OH group,
R₂ represents hydrogen, a linear or branched C₁₋₆ alkyl group optionally containing a silicon atom, a cyano group, a C₁₋₆ alkylsulphonyl group or a phenylsulphonyl group,
R₃ represents a linear or branched C₁₋₆ alkyl group optionally containing a silicon atom or a phenyl group that can form a bicycle and optionally substituted with one or two radicals R₄,
or R₂ and R₃ together form a monocyclic, bicyclic or tricyclic C₂₋₁₀ hydrocarbon residue optionally interrupted with one or more nitrogen, sulphur and oxygen atoms and possibly containing another carbonyl, and optionally substituted with a linear or branched C₁-C₈ alkylsulphonamide group, and optionally containing a silicon atom or an amino acid function; on condition that, when n=1, R₂ and R₃ do not form a camphor nucleus.

2. Insoluble organic compound of formula (I) in which n=1 according to claim 1, characterized in that it is chosen from styryl ketones, benzylidene cineoles, benzylidene chromanones, benzylidene thiochromanones, benzylidene quinuclidinones, benzylidene cycloalkanones, benzylidene hydantoins, benzylidene indanones, benzylidene tetralones, benzylidene furanones, benzylidene benzofuranones, benzylidene indanediones, benzylidene benzothiofuranones, benzylidene barbiturics, benzylidene pyrazolones, benzylidene imidazolones, chalcones and benzylidene ones.

3. Insoluble organic compound of formula (I) in which n=2 according to claim 1, characterized in that it is chosen from phenylenebis(methylidene-nor-camphors), phenylenebis(methylidenecamphors), phenylenebis(methylidenecamphorsulphonamides), phenylenebis(methylidene cineoles), phenylenebis(methylidene ketotricyclodecanes), bis(phenylene alkylene ketones), phenylenebis(methylidene furanones) and phenylenebis(methylidene quinuclidinones).

4. Insoluble organic compound of formula (II) according to claim 1, characterized in that it is chosen from bis(benzylidene cycloalkanones) and gamma-pyrones.

5. Insoluble organic compound according to any one of the preceding claims, characterized in that it is in the form of particles with an average size of between 10 nm and 2 μm and preferably between 20 nm and 2 μm.

6. Cosmetic composition containing at least one insoluble organic compound for screening out UV radiation according to any one of the preceding claims, in a cosmetically acceptable support.

7. Cosmetic composition according to claim 6, characterized in that the fraction of the insoluble organic compound for screening out UV radiation is between 0.1% and 15% by weight and preferably between 0.2% and 10% by weight, relative to the total weight of the cosmetic composition.

8. Cosmetic composition according to either of claims 6 and 7, characterized in that it also contains one or more organic sunscreens that are active in the UV-A and/or UV-B range, other than those according to claims 1 to 5.

9. Cosmetic composition according to claim 8, characterized in that the organic sunscreens are chosen from the following: ethylhexyl salicylate, butyl methoxydibenzoylmethane, ethylhexyl methoxycinnamate, octocrylene, phenylbenzimidazolesulphonic acid, terephthalylidenedicamphorsulphonic acid, benzophenone-3, benzophenone-4, benzophenone-5, 4-methylbenzylidenecamphor, benzimidazilate, anisotriazine, ethylhexyltriazone, diethylhexylbutamidotriazone, 2,4,6-tris(diisobutyl 4′-aminobenzalmalonate) s-triazine, methylenebis(benzotriazolyl)tetramethylbutylphenol, drometrizole trisiloxane, and mixtures thereof.

10. Cosmetic composition according to any one of claims 6 to 9, characterized in that it also contains one or more mineral pigments.

11. Cosmetic composition according to claim 10, characterized in that the mineral pigments are chosen from nanopigments of metal oxides such as titanium oxide, iron oxide, zinc oxide, zirconium oxide and cerium oxide, that are optionally coated, and mixtures thereof.

12. Cosmetic composition according to claim 11, characterized in that the nanopigments have a mean particle size of between 5 nm and 100 nm and preferably between 10 nm and 50 nm.

13. Cosmetic composition according to any one of claims 6 to 12, characterized in that it also contains at least one agent for artificially tanning and/or browning the skin.

14. Cosmetic composition according to any one of claims 6 to 13, characterized in that it also contains one or more formulation adjuvants chosen from fatty substances, physiologically acceptable organic solvents, thickeners, antioxidants, opacifiers, stabilizers, antifoams, fragrances, preserving agents, fillers, sequestering agents, propellants, pH modifiers and colorants, and mixtures thereof.

15. Cosmetic composition according to any one of claims 6 to 14, characterized in that it also contains one or more cosmetic active principles chosen from vitamins, softeners, hydroxy acids, moisturizers, emollients, free-radical scavengers, substance P antagonists, anti-inflammatories and mixtures of these compounds.

16. Cosmetic composition according to any one of claims 6 to 15, characterized in that it constitutes a cosmetic composition for protecting the skin or the hair, an antisun composition or a makeup product.

17. Use of one or more compounds according to any one of claims 1 to 5 for protecting materials that are sensitive to UV radiation, in particular solar radiation.

18. Composition for protecting a material that is sensitive to UV radiation, in particular solar radiation, characterized in that it comprises an effective amount of at least one compound according to any one of claims 1 to 5.

19. Use of one or more compounds according to any one of claims 1 to 5, for the preparation of a medicinal product for preventing the harmful effects of UV radiation.

20. Non-therapeutic process for protecting the skin or the hair against ultraviolet radiation, in particular solar radiation, characterized in that it consists in applying to the skin or the hair an effective amount of a cosmetic composition according to any one of claims 6 to 16, or of a compound according to any one of claims 1 to 5.

21. Non-therapeutic process for controlling the variation in the colour of the skin caused by UV radiation, characterized in that it consists in applying to the skin an effective amount of a cosmetic composition according to any one of claims 6 to 15, or of a compound according to any one of claims 1 to 5.
